(19) United States
(12) Patent Application Publication
(10) Pub. No.: US 2017/0135617 A1
(43) Pub. Date: May 18, 2017

(54) OPTOELECTRONIC MODULES OPERABLE TO DISTINGUISH BETWEEN SIGNALS INDICATIVE OF REFLECTIONS FROM AN OBJECT OF INTEREST AND SIGNALS INDICATIVE OF A SPURIOUS REFLECTION

(71) Applicant: Heptagon Micro Optics Pte. Ltd., Singapore (SG)

(72) Inventors: Jukka Alasirniö, Jääli (FI); Tobias Senn, Zurich (CH); Mario Cesana, Au (CH); Hartmut Rudmann, Jona (CH); Markus Rossi, Jona (CH); Peter Roentgen, Thalwil (CH); Daniel Pérez Calero, Zurich (CH); Bassam Hallal, Thalwil (CH); Jens Geiger, Thalwil (CH)

(21) Appl. No.: 15/325,811

(22) PCT Filed: Jul. 13, 2015
(86) PCT No.: PCT/SG2015/050211

§ 371 Date: Jan. 12, 2017

Related U.S. Application Data
(60) Provisional application No. 62/024,040, filed on Jul. 14, 2014.
(60) Provisional application No. 62/051,128, filed on Sep. 16, 2014.

(51) Int. Cl. A61B 5/1455; A61B 5/024; G01J 1/42; G01S 17/48; G01S 7/497

(52) U.S. Cl. CPC A61B 5/14551; G01S 17/48; G01S 7/497; G01J 1/4204; A61B 5/02416; G01S 2007/4975

(57) ABSTRACT

The present disclosure describes modules operable to perform optical sensing. The module can be operable to distinguish between signals indicative of reflections from an object or interest and signals indicative of a spurious reflection such as from a smudge (i.e., a blurred or smeared mark) on the host device's cover glass. Signals assigned to reflections from the object of interest can be used to for various purposes, depending on the application (e.g., determining an object's proximity, a person's heart rate or a person's blood oxygen level).

### TECHNICAL FIELD

The present disclosure relates to modules that provide optical signal detection.

### BACKGROUND

Some handheld computing devices such as smart phones can provide a variety of different optical functions such as one-dimensional (1D) or three-dimensional (3D) gesture detection, 3D imaging, proximity detection, ambient light sensing, and/or front-facing two-dimensional (2D) camera imaging.

Proximity detectors, for example, can be used to detect the distance to (i.e., proximity of) an object up to distances on the order of about one meter. In some cases, a smudge (e.g., fingerprint) on the transmissive window or cover glass of the host device can produce a spurious proximity signal, which may compromise the accuracy of the proximity data collected.

### SUMMARY

The present disclosure describes optoelectronic modules operable to distinguish between signals indicative of reflections from an object interest and signals indicative of a spurious reflection. Modules also are described in which particular light projectors in the module can serve multiple functions (e.g., can be used in more than one operating mode).

For example, in one aspect, a module is operable to distinguish between signals indicative of an object of interest and signals indicative of a spurious reflection, for example from a smudge (i.e., a blurred or smeared mark) on the host device's cover glass. The module can include a light projector operable to project light out of the module, and an image sensor including spatially distributed light sensitive components (e.g., pixels of a sensor) that are sensitive to a wavelength of light emitted by the light projector. The module includes processing circuitry operable to read signals from the spatially distributed light sensitive components of the image sensor and to assign peak signals associated, respectively, with particular ones of the light sensitive components either to a reflection from an object of interest (e.g., outside the host device) or to a spurious reflection (e.g., resulting from a smudge on a transmissive window of a host device).

In some implementations, a single module can be used for one or more of the following applications: proximity sensing, heart rate monitoring and/or reflectance pulse oximetry applications. In each case, processing circuitry can distinguish between spurious signals (e.g., signals indicative of reflections caused by a smudge on a cover glass) and signals of interest (e.g., signals indicative of reflections from an object whose proximity is to be determined, or a person's finger or other body part, in the case of heart rate monitoring and/or reflectance pulse oximetry applications). The signals of interest then can be processed, depending on the application, to obtain a distance to an object, to determine a person's blood oxygen level or to determine a person's heart rate. In some implementations, the module can be used for stereo imaging in addition to one or more of the foregoing applications. The addition of a light projector that provides structured light can be advantageous, for example, in some imaging applications as well. In some implementations, a particular light projector can serve multiple functions. For example, in some cases, a light projector that is operable to emit red light can be used when the module is operating in a flash mode or when the module is operating in a reflectance pulse oximetry mode.

When used for proximity sensing applications, some implementations can provide enhanced proximity detection. For example, some implementations include more than one light projector to project light out of the module toward an object of interest. Likewise, some implementations may include more than one optical channel. Such features can, in some cases, help improve accuracy in the calculation of the object's proximity.

In another aspect, a proximity sensing module includes a first optical channel disposed over an image sensor having spatially distributed light sensitive components. A first light projector is operable to project light out of the module. There is a first baseline distance between the first light projector and the optical axis of the channel. A second light projector is operable to project light out of the module. There is a second baseline distance between the second light projector and the optical axis. An image sensor including spatially distributed light sensitive components that are sensitive to a wavelength of light emitted by the first light projector and a wavelength of light emitted by the second light projector. Processing circuitry is operable to read and process signals from the spatially distributed light sensitive components of the image sensor.

In some cases, the processing circuitry is operable to identify particular ones of the spatially distributed light sensitive components that sense peak signals based on light emitted by the first and second light projectors, and to determine a proximity to an object outside the module based at least in part on positions of the particular ones of the spatially distributed light sensitive components. In some instances, the first and second baseline distances differ from one another. Such features can, in some cases, help increase the range of proximities that can be detected.

In some cases, a particular optical channel and its associated spatially distributed light sensitive components can be used for other functions in addition to proximity sensing. For example, the same optical channel(s) may be used for proximity sensing as well as imaging or gesture recognition. In some cases, different imagers in the module or different parts of the light sensitive components can be operated dynamically in different power modes depending on the optical functionality that is required for a particular application. For example, a high-power mode may be used for 3D stereo imaging, whereas a low-power mode may be used for proximity and/or gesture sensing. Thus, in some cases, signals from pixels associated, respectively, with the different imagers can be read and processed selectively to reduce power consumption.

The modules may include multiple light sources (e.g., vertical cavity surface emitting lasers (VCSELs)) that generate coherent, directional, spectrally defined light emission. In some applications (e.g., 3D stereo matching), a high-power light source may be desirable, whereas in other applications (e.g., proximity or gesture sensing), a low-power light source may be sufficient. The modules can include both high-power and low-power light sources, which selectively can be turned on and off. By using the low-power light source for some applications, the module's overall power consumption can be reduced.

Thus, a single compact module having a relatively small footprint can provide a range of different imaging/sensing functions and can be operated, in some instances, in either a high-power mode or a low-power mode. In some cases, enhanced proximity sensing can be achieved. In some cases, by using different areas of the same image sensor for various functions, the number of small openings in the front casing of the smart phone or other host device can be reduced.

Other aspects, features and advantages will be readily apparent from the following detailed description, the accompanying drawings, and the claims.

### BRIEF DESCRIPTION OF THE DRAWINGS

FIG. 1 illustrates a side view of an example of a module for proximity sensing.

FIG. 2 illustrates additional details of the proximity sensor in the module of FIG. 1.

FIG. 3 illustrates various parameters for calculating proximity of an object using triangulation.

FIGS. 4A and 4B illustrate an example of proximity sensing using multiple optical channels.

FIGS. 5A and 5B illustrate an example of a module that includes multiple light projectors for use in proximity sensing.

FIG. 6 illustrates an example of a module that includes multiple light projectors having different baselines.

FIGS. 7A-7C illustrate examples of a module including a light projector that projects light at an angle.

FIG. 7D illustrates a side view of an example of a module that has a tilted field-of-view for proximity detection; FIG. 7E is a top view illustrating an arrangement of features of FIG. 7D; FIG. 7F is another side view of the module illustrating further features.

FIG. 8 illustrates an example of a module using a structured light pattern for proximity sensing.

FIG. 9 illustrates an example of a module using a structured light pattern for imaging.

FIG. 10 illustrates an example of a module using ambient light for imaging.

FIG. 11 illustrates an example of a module that includes a high-resolution primary imager and one or more secondary imagers.

FIGS. 12A-12H illustrate various arrangements of modules in which one or more imagers share a common image sensor.

FIGS. 13A-13C illustrate various arrangements of modules in which a primary imager and one or more secondary imagers have separate image sensors.

FIGS. 14A-14C illustrate various arrangements of modules that include an autofocus assembly.

FIG. 15 illustrates an arrangement of a module that includes an ambient light sensor.

FIGS. 16A-16E illustrate examples of modules for reflectance pulse oximetry and/or heart rate monitoring applications.

FIGS. 17A and 17B illustrate examples of modules including a multi-functional red light projector that can be used in a reflectance pulse oximetry mode, a flash mode and/or an indicator mode.

### DETAILED DESCRIPTION

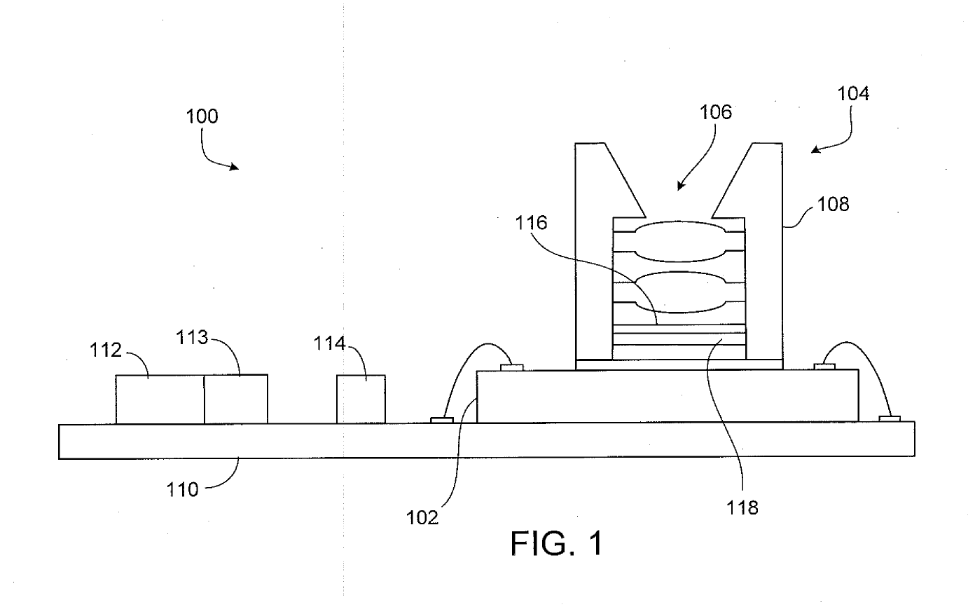

As illustrated in FIG. 1, an optical module 100 is operable to provide proximity sensing (i.e., detecting the presence of an object and/or determining its distance). The module 100 includes an image sensor 102 that has photosensitive regions (e.g., pixels) that can be implemented, for example, on a single integrated semiconductor chip (e.g., a CCD or CMOS sensor). The imager 104 includes a lens stack 106 disposed over the photosensitive regions of the sensor 102. The lens stack 106 can be placed in a lens barrel 108. The sensor 102 can be mounted on a printed circuit board (PCB) 110 or other substrate. Electrical connections (e.g., wires or flip-chip type connections) can be provided from the sensor 102 to the PCB 110. Processing circuitry 112, which also can be mounted, for example, on the PCB 110, can read and process data from the imager 104. The processing circuitry 112 can be implemented, for example, as one or more integrated circuits in one or more semiconductor chips with appropriate digital logic and/or other hardware components (e.g., read-out registers; amplifiers; analog-to-digital converters; clock drivers; timing logic; signal processing circuitry; and/or a microprocessor). The processing circuitry 112 is, thus, configured to implement the various functions associated with such circuitry.

The module 100 also includes a light projector 114 such as a laser diode or vertical cavity surface emitting laser that is operable to emit coherent, directional, spectrally defined light emission. The light projector 114 can be implemented, for example, as a relatively low-power VCSEL (e.g., output power in the range of 1-20 mW, preferably about 10 mW) that can project infra-red (IR) light. The light projector 114 used for proximity sensing need not simulate texture and, therefore, can simply project an optical dot onto an object, whose distance or presence is to be detected based on light reflected from the object. In some implementations, the light projector 114 is operable to emit a predetermined narrow range of wavelengths in the IR part of the spectrum. The light projector 114 in some cases may emit light in the range of about 850 nm+10 nm, or in the range of about 830 nm+10 nm, or in the range of about 940 nm+10 nm. Different wavelengths and ranges may be appropriate for other implementations. The light emitted by the projector 114 may be reflected, for example, by an object external to the host device (e.g., a smart phone) such that the reflected light is directed back toward the image sensor 102.

In the illustrated module of FIG. 1, the imager 104 includes a band-pass filter 116 disposed, for example, on a transmissive window which may take the form of a cover glass 118. The band-pass filter 116 can be designed to filter substantially all IR light except for wavelength(s) of light emitted by the light projector 114 and can be implemented, for example, as a dielectric-type band-pass filter.

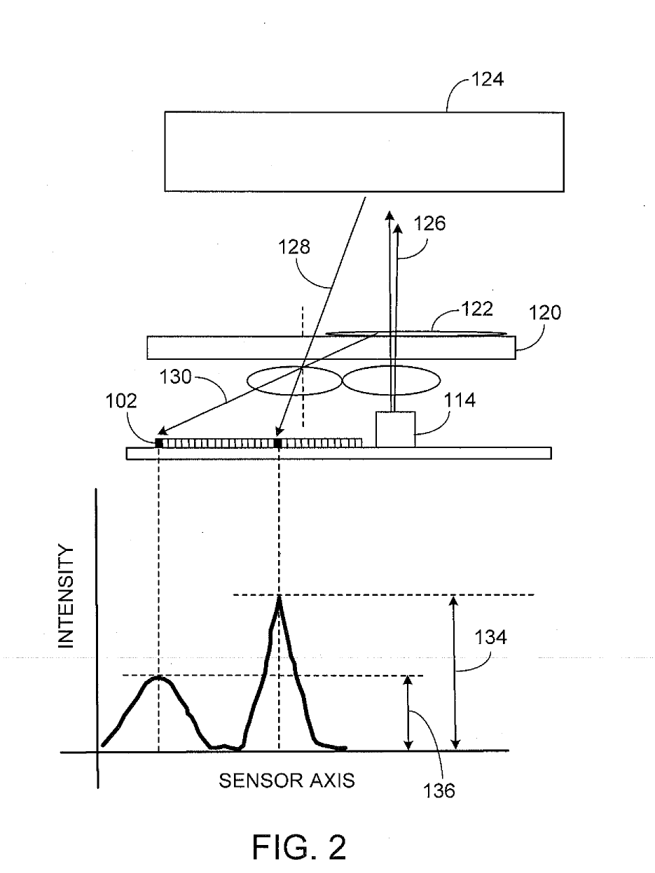

The module 100 can, in some cases, provide enhanced proximity sensing. For example, use of a VCSEL as the light projector 114 can provide coherent, more directional, and spectrally defined light emission than a LED. Further, as the image sensor 102 is composed of spatially distributed light sensitive components (e.g., pixels of a CMOS sensor), peaks in the detected intensity can be assigned by the processing circuitry 112 either to an object 124 of interest external to the host device or to a spurious reflection such as from a smudge 122 (i.e., a blurred or smeared mark) on the transmissive window 120 of the host device (see FIG. 2).

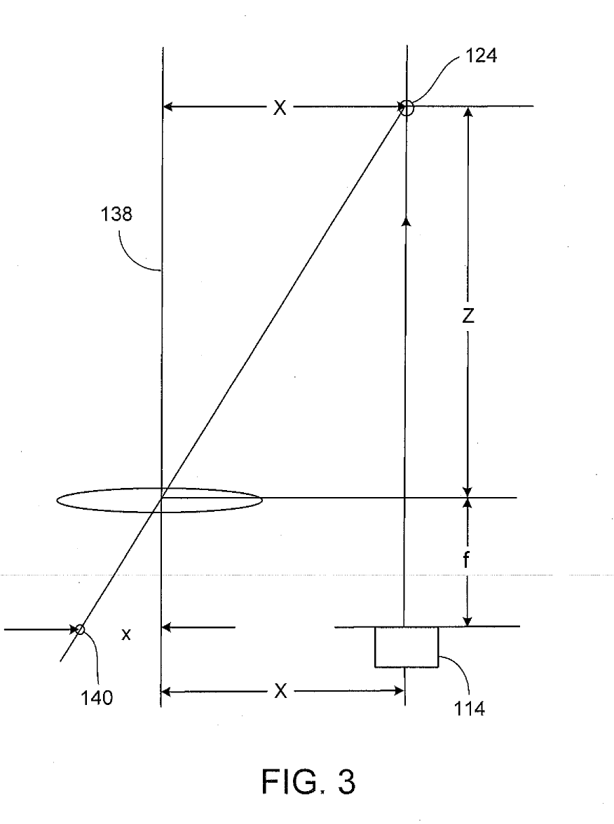

As shown in the example of FIG. 2, when light 126 is emitted from the light projector 114 toward an object 124 (e.g., a human ear), some light 128 is reflected by the object 124 and detected by the imager sensor 102, and some light 130 is reflected by a smudge 122 on the transmissive window 120 of the host device (e.g., the cover glass of a smart phone) and detected by the image sensor 102. The reflected light 128, 130 can be detected by the pixels of the image sensor 102 at different intensities, as illustrated in the graphical depiction in the lower part of FIG. 2. The intensity of reflection and the distribution (i.e., shape of the curve) may be significantly different for the object 124 and the smudge 122. Thus, the processing circuitry 112 can assign one of the peaks (e.g., peak 134), based on predetermined criteria, as indicative of the object's proximity (i.e., distance), and another one of the peaks (e.g., peak 136) can be assigned, based on predetermined criteria, as indicative of the smudge 122 (or some of other spurious reflection). The processing circuitry 112 than can use a triangulation technique, for example, to calculate the distance “Z” of the object 124. The triangulation technique can be based, in part, on the baseline distance “X” between the light projector 114 and the optical axis 138 of the optical channel, and the distance “x” between the pixel 140 at which the peak 134 occurs and the optical axis 138 of the optical channel. The distances “x” and “X” can be stored or calculated by the processing circuitry 112. Referring to FIG. 3:

$$Z = \frac{X \cdot f}{x}$$

where “f” is the focal length of the lens stack, and Z is the proximity (i.e., the distance to the object 124 of interest). As the measured intensities are spatially defined and can be assigned either to the object 124 or to the smudge 122, the measured optical intensity associated with the object 124 can be correlated more accurately to distance. Such proximity detection can be useful, for example, in determining whether a user has moved a smart phone or other host device next to her ear. If so, in some implementations, control circuitry in the smart phone may be configured to turn off the display screen to save power. In some instances, the processing circuitry 112 can use the distance between the spurious reflection (e.g., the smudge signal) and the object signal as further input to correct for the measured intensity associated with the object 124.

In some cases, instead of, or in addition to, calculating the proximity of the object 124 using a triangulation technique, the intensity of the peak 134 associated with the object 124 can be correlated to a proximity (i.e., distance) using a look-up table or calibration data stored, for example, in memory associated with the processing circuitry 112.

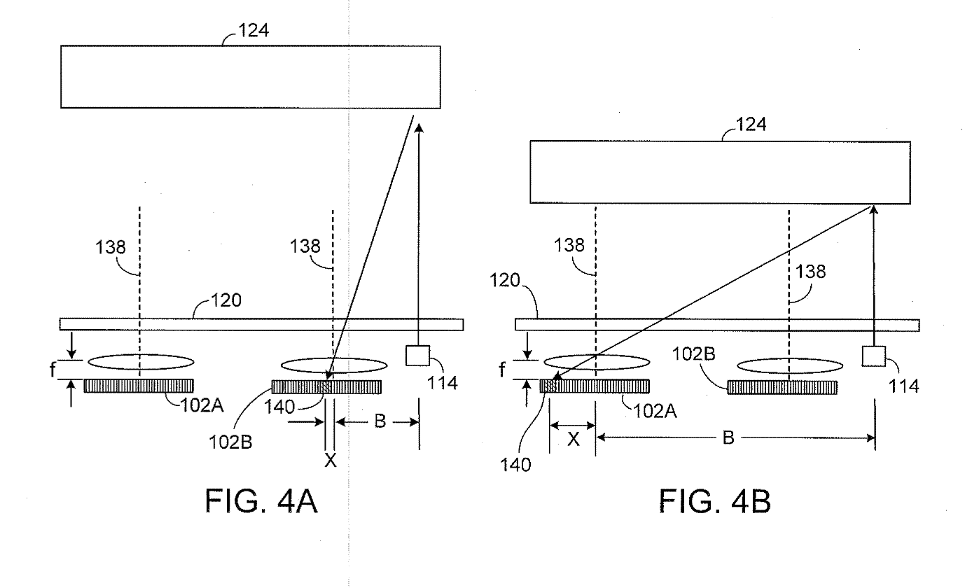

In some implementations, it may be desirable to provide multiple optical channels for proximity sensing. Thus, data can be read and processed from more than one imager 104 (or an imager having two or more optical channels) so as to expand the depth range for detecting an object. For example, data detected by pixels 102B associated with a first optical channel may be used to detect the proximity of an object 124 at a position relatively far from the transmissive window 120 of the host device (FIG. 4A), whereas data detected by pixels 102A in a second channel may be used to detect the proximity of an object 124 at a position relatively close to the transmissive window 120 (FIG. 4B). Each channel has its own baseline “B” (i.e., distance from the light projector 114 to the channel's optical axis 138) that differs from one channel to the next.

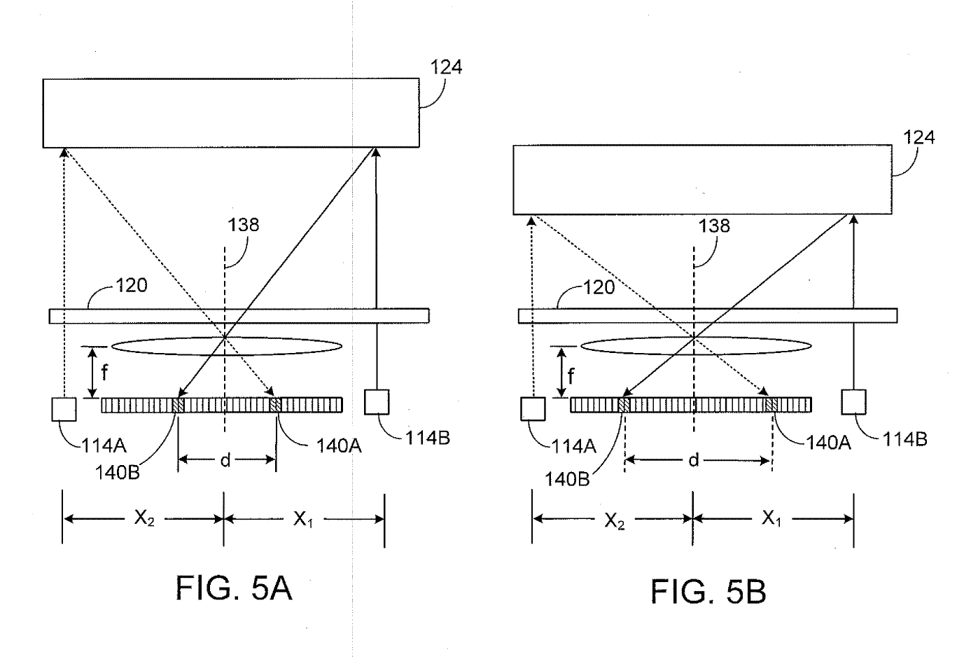

As shown in FIGS. 5A and 5B, in some instances, it can be advantageous to provide multiple (e.g., two) light projectors 114A, 114B for proximity sensing using a single optical channel. Each of the light projectors 114A, 114B can be similar, for example, to the light projector 114 described above. Light emitted by the light projectors 114A, 114B and reflected by the object 124 can be sensed by the image sensor. The processing circuitry 112 can determine and identify the pixels 140A, 140B at which peak intensities occur. The distance “d” between the two pixels 140A, 140B corresponds to the proximity “Z” of the object 124. In particular, the distance “d” is inversely proportional to the proximity “Z”:

$$Z = \frac{f \cdot ( {X}_{1} + {X}_{2} )}{d} ,$$

where “f” is the focal length of the lens stack, “X₁” is the distance (i.e., baseline) between the first light projector 114B and the optical axis 138 of the optical channel, and “X₂” is the distance (i.e., baseline) between the second light projector 114A and the optical axis 138. In general, the greater the value of “Z,” the smaller will be the distance “d” between the pixels 140A, 140B. Conversely, the smaller the value of “Z,” the greater will be the distance “d” between the pixels 140A, 140B. Thus, since the value of “d” in FIG. 5A is smaller than the value of “d” in FIG. 5B, the processing circuitry 112 will determine that the object 124 is further away in the scenario of FIG. 5A than in the scenario of FIG. 5B.

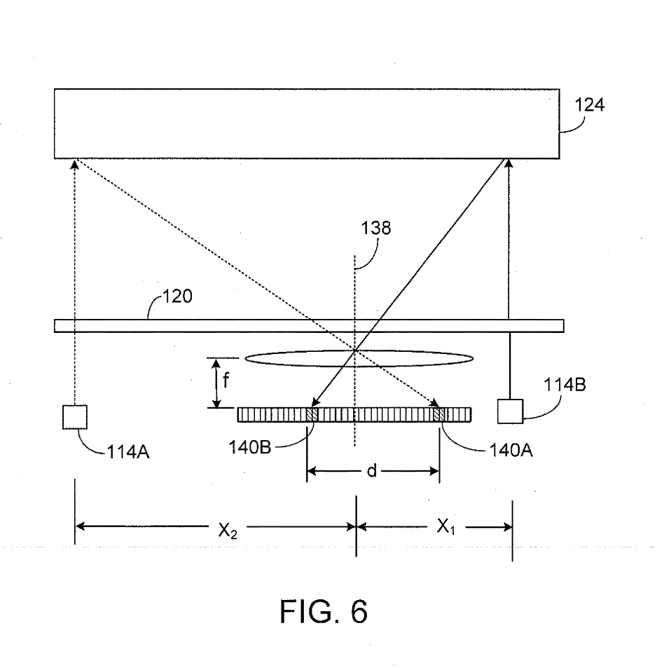

In the examples of FIGS. 5A and 5B, it is assumed that the baselines for the two light projectors 114A, 114B (i.e., the values of X₁ and X₂) are substantially the same as one another. However, as illustrated in FIG. 6, in some implementations, the baselines may differ from one another. For example, the light projector 114A having the larger baseline (X₂) may be used for detecting the proximity of a relatively distant object 124, whereas the smaller baseline (X₁) may be used for detecting the proximity of a relatively close object. Providing multiple light projectors having different baselines can help increase the overall range of proximity distances that can be detected by the module (i.e., each baseline corresponds to a different proximity range). In some implementations, the same image sensor 102 is operable for proximity sensing using either of the light projectors 114A, 114B. In other implementations, a different image sensor is provided for each respective light projector 114A, 114B.

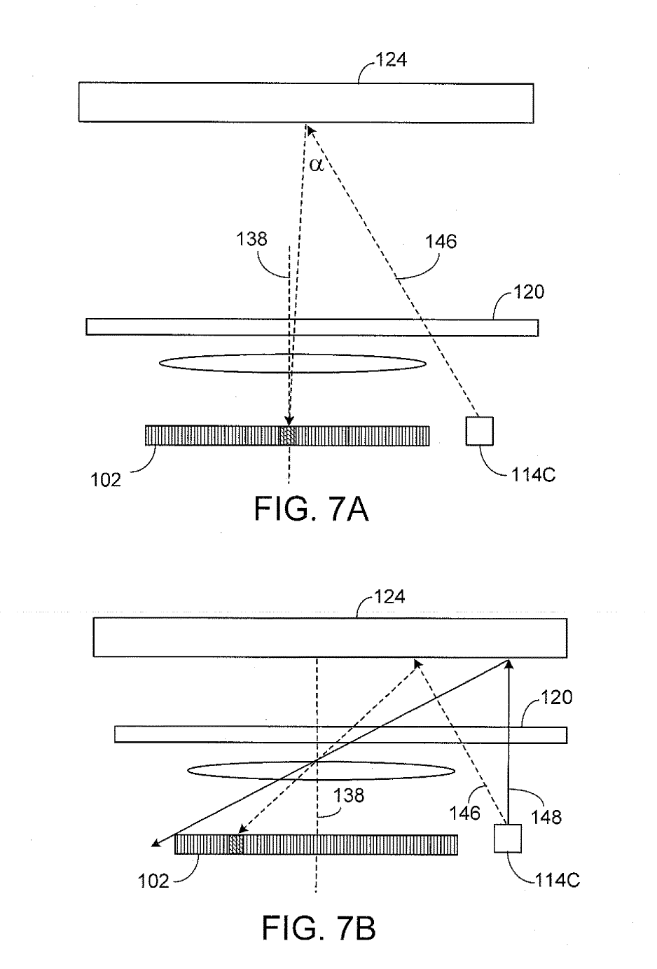

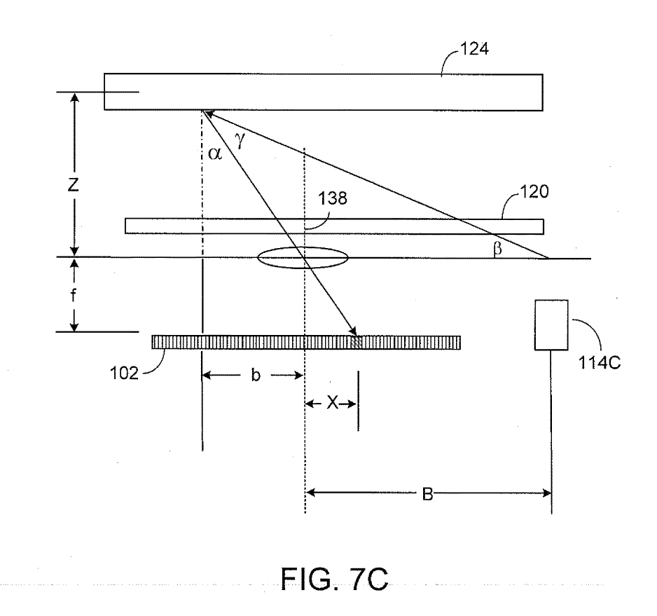

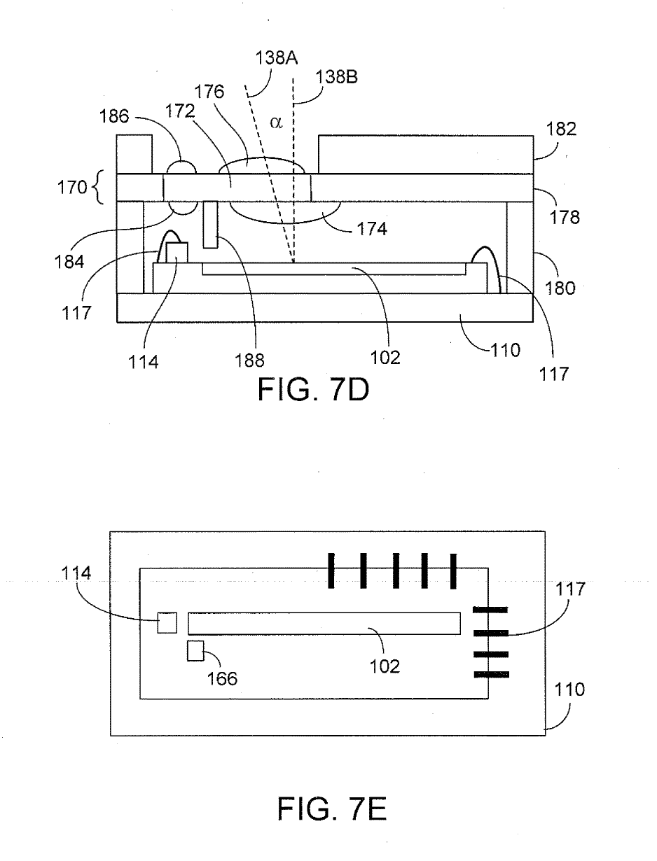

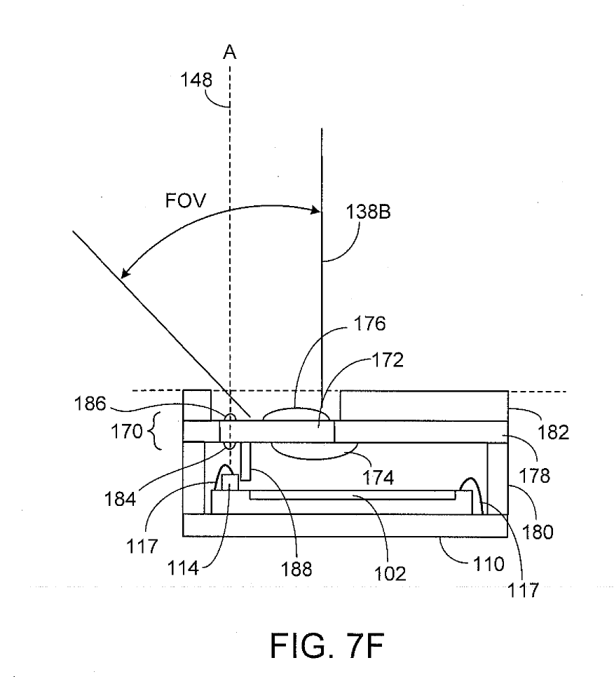

In some implementations, as illustrated in FIG. 7A, the module includes a light projector 114C that is operable to project collimated light at an angle (I) relative to the channel's optical axis 138, where I=90°-β. The angle (β), in some cases, is in the range 20°≦β<90°, although preferably it is in the range 45°≦β<90°, and even more preferably in the range 80°≦β<90°. The module 114C can be provided in addition to, or as an alternative to, a light projector that projects collimated light substantially parallel to the channel's optical axis 138. As shown in FIG. 7B, in some cases, collimated light 148 projected substantially parallel to the optical axis 138 may not be detected by the image sensor 102 when the light is reflected by the object 124. Thus, providing a light projector 114C that emits collimated light at an angle relative to the optical axis 138 can help expand the range of distances that can be detected for proximity sensing. As shown in the example of FIG. 7C, the proximity (“Z”) can be calculated, for example, by the processing circuitry 112 in accordance with the following equation:

$$Z = ( \frac{sin β}{sin γ} B sin α ) ( \frac{f}{x} )$$

In some implementations, instead of (or in addition to) providing an emitter that emits light at an angle with respect to the emission channel's optical axis, the proximity detection module has a tilted field-of-view (FOV) for the detection channel. An example is illustrated in FIGS. 7D, 7E and 7F, which show a module that includes a light emitter 114 and an image sensor 102. An optics assembly 170 includes a transparent cover 172 surrounded laterally by a non-transparent optics member 178. A spacer 180 separates the optics member 178 from the PCB 110. Wire bonds 117 can couple the light emitter 114 and image sensor 102 electrically to the PCB 110.

The optics assembly 170 includes one or more beam shaping elements (e.g., lenses 174, 176) on the surface(s) of the transparent cover 172. The lenses 174, 176 are arranged over the image sensor 102 such that the optical axis 138A of the detection channel is tilted at an angle (a) with respect to a line 138B that is perpendicular to the surface of the image sensor 102. The lenses 174, 176 may be offset with respect to one another. In some implementations, the angle α is about 30°+10°. Other angles may be appropriate in some instances. A baffle 182 can be provided to reduce the likelihood that stray light will be detected and to protect the optics assembly 170. As illustrated in FIG. 7F, the resulting FOV for the detection channel can, in some cases, facilitate proximity detection even for objects very close to the object-side of the module (e.g., objects in a range of 0-30 cm from the module). In some implementations, the resulting FOV is in the range of about 40°+10°. Other values may be achieved in some implementations.

Although the light beam emitted by the emitter 114 may have a relatively small divergence (e.g., 10°-20°), in some cases, it may be desirable to provide one or more beam shaping elements (e.g., collimating lenses 184, 186) on the surface(s) of the transparent cover 172 so as to reduce the divergence of the outgoing light beam even further (e.g., total divergence of 2°-3°). Such collimating lenses may be provided not only for the example of FIGS. 7D-7F, but for any of the other implementations described in this disclosure as well. Further in some implementations, a non-transparent vertical wall 188 is provided to reduce or eliminate optical cross-talk (i.e., to prevent light emitted by the emitter 106 from reflecting off the collimating lenses 184, 186 and impinging on the image sensor 102). The wall 188 can be implemented, for example, as a projection from the imager-side of the transparent cover 172 and may be composed, for example, of black epoxy or other polymer material. In some implementations, a single module can provide proximity sensing as well as ambient light sensing. This can be accomplished, as shown for example in FIG. 7E, by including in the module an ambient light sensor (ALS) 166 and one or more beam shaping elements (e.g., lenses) to direct ambient light onto the ALS. Preferably, the lenses for the light ALS 166 provide a FOV of at least 120°. In some embodiments, the overall dimensions of the module can be very small (e.g., 1.5 mm (height)×3 mm (length)×2 mm (width)).

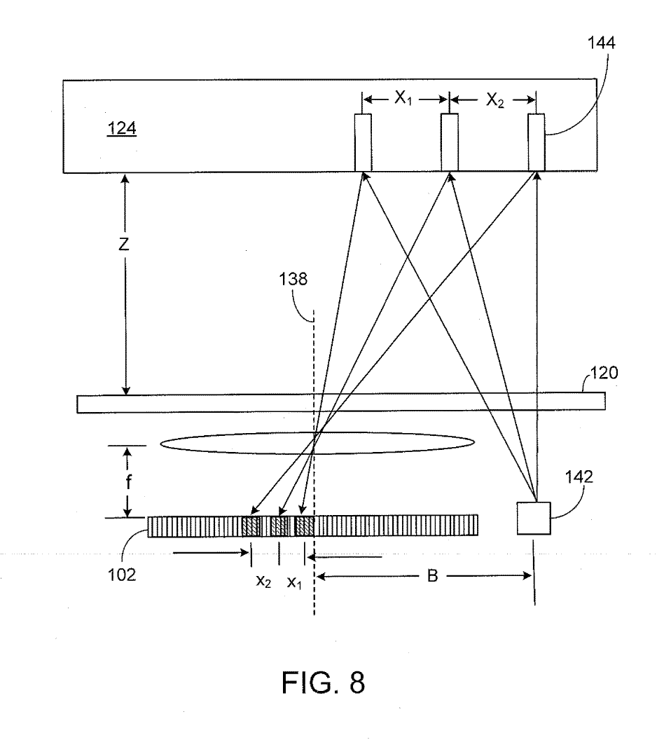

In some cases, as illustrated by FIG. 8, the module includes a light projector 142 operable to project structured light 144 (e.g., a pattern of stripes) onto an object 124. For example, a high-power laser diode or VCSEL (e.g., output power in the range of 20-500 mW, preferably about 150 mW), with appropriate optics, can be used to emit a predetermined narrow range of wavelengths in the IR part of the spectrum. The light projector 142 in some cases may emit light in the range of about 850 nm+10 nm, or in the range of about 830 nm+10 nm, or in the range of about 940 nm+10 nm. Different wavelengths and ranges may be appropriate for other implementations. The FOV of the imager 102 and the FOV of the light projector 142 should encompass the object 124. The structured light projector 142 can be provided in addition to, or as an alternative to, the light projector 114 that emits a single beam of collimated light.

The structured light emitted by the light projector 142 can result in a pattern 144 of discrete features (i.e., texture) being projected onto an object 124 external to the host device (e.g., a smart phone) in which the module is located. Light reflected by the object 124 can be directed back toward the image sensor 102 in the module. The light reflected by the object 124 can be sensed by the image sensor 102 as a pattern and may be used for proximity sensing. In general, the separation distances x₁ and x₂ in the detected pattern change depending on the distance (i.e., proximity) of the object 124. Thus, for example, assuming that the focal length (“f”), the baseline distance (“B”) between the light projector 142 and the channel's optical axis 138, and the angle of emission from the structured light source 142 are known, the proximity (“Z”) can be calculated by the processing circuitry 112 using a triangulation technique. The values of the various parameters can be stored, for example, in memory associated with the processing circuitry 112. Alternatively, the proximity can be determined from a look-up table stored in the module's memory. In some cases, the proximity of the object 124 can be determined based on a comparison of the measured disparity xᵢ and a reference disparity, where a correlation between the reference disparity and distance is stored by the module's memory.

In some implementations, distances may be calculated by projected structured light using the same triangulation method as the non-structured light projector. The structured light emitter also can be useful for triangulation because it typically is located far from the imager (i.e., a large baseline). The large baseline enables better distance calculation (via triangulation) at longer distances.

In some implementations, the optical channel that is used for proximity sensing also can be used, for other functions, such as imaging. For example, signals detected by pixels of the image sensor 102 in FIG. 1 can be processed by the processing circuitry 112 so as to generate an image of the object 124. Thus each optical channel in any of the foregoing modules can be used, in some cases, for both proximity sensing and imaging.

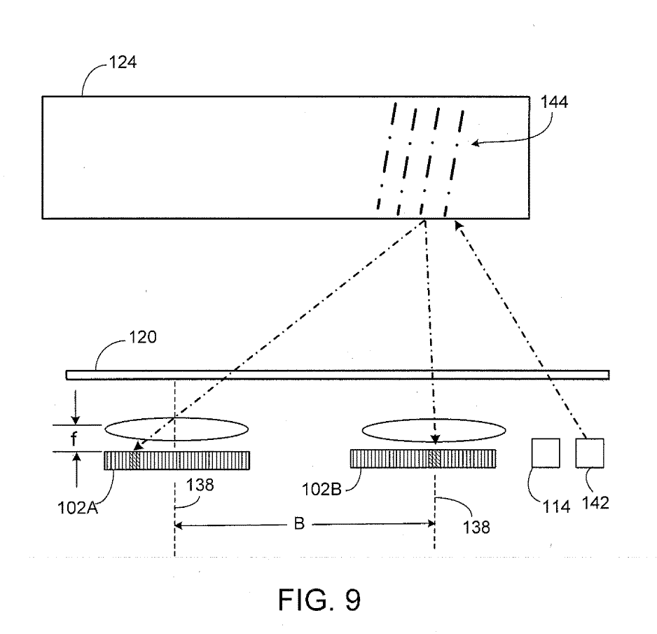

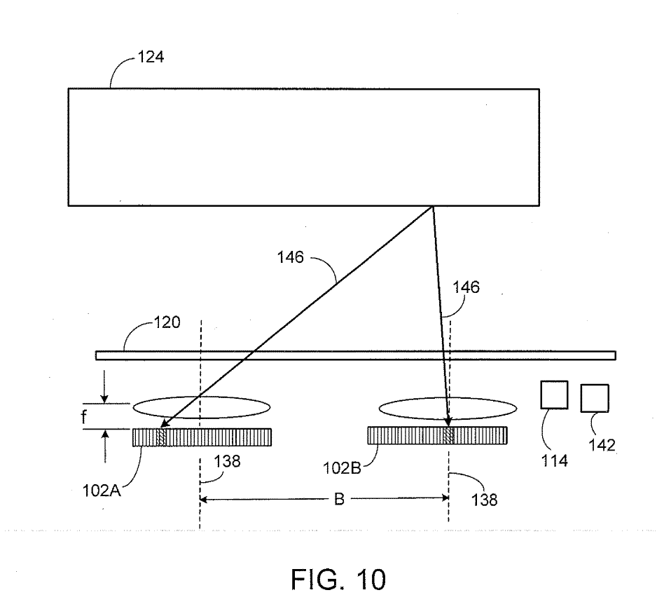

As noted above, some implementations include two or more optical channels each of which is operable for use in proximity sensing. In some cases, the different channels may share a common image sensor, whereas in other cases, each channel may be associated with a different respective image sensor each of which may be on a common substrate. In implementations where multiple channels are used to acquire image data, the processing circuitry 112 can combine depth information acquired from two or more of the channels to generate three-dimensional (3D) images of a scene or object. Further, in some instances, as illustrated by FIG. 9, a light source (e.g., a VCSEL or laser diode) 142 can be used to project a structured IR pattern 144 onto a scene or object 124 of interest. Light from the projected pattern 144 is reflected by the object 124 and sensed by different imagers 102A, 102B for use in stereo matching to generate the 3D image. In some cases, the structured light provides additional texture for matching pixels in stereo images. Signals from the matched pixels also can be used to improve proximity calculations. Further, in some instances, as indicated by FIG. 10, ambient light 146 reflected from the object 124 can be used for the stereo matching (i.e., without the need to project structured light 144 from the light source 142).

In some implementations, the structured pattern 144 generated by the light source 142 can be used for both imaging as well as proximity sensing applications. The module may include two different light projectors, one of which 142 projects a structured pattern 144 used for imaging, and a second light projector 114 used for proximity sensing. Each light projector may have an optical intensity that differs from the optical intensity of the other projector. For example, the higher power light projector 142 can be used for imaging, whereas the lower power light projector 114 can be used for proximity sensing. In some cases, a single projector may be operable at two or more intensities, where the higher intensity is used for imaging, and the lower intensity is used for proximity sensing.

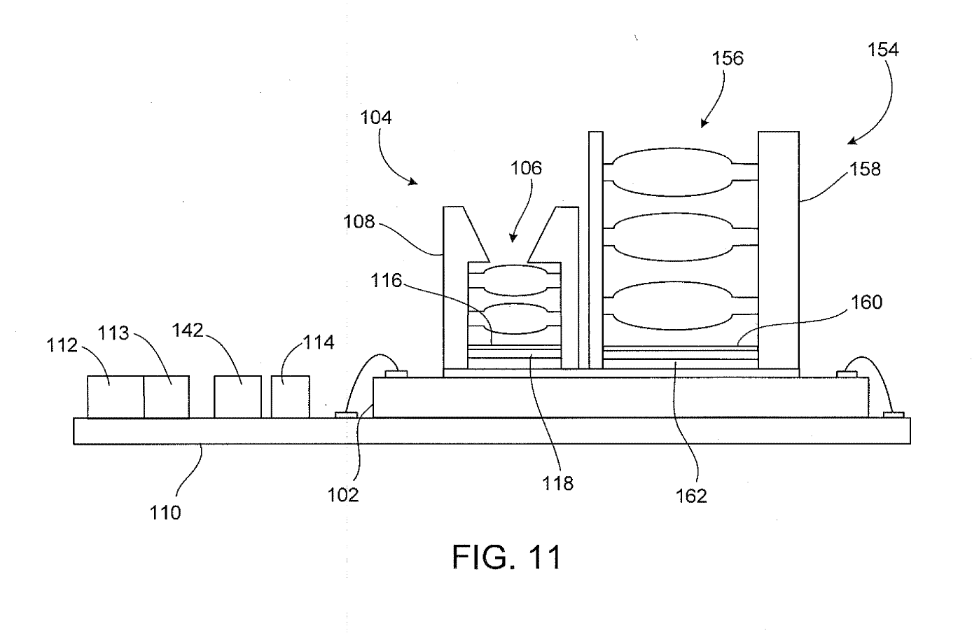

To enhance imaging capabilities, as shown in FIG. 11, some implementations of the module include a primary high-resolution imager 154 (e.g., 1920 pixels×1080 pixels) in addition to one or more secondary imagers 104 as described above. The primary imager 154 is operable to collect signals representing a primary two-dimensional (2D) image. The secondary imagers 104, which can be used for proximity sensing as described above, also can be used to provide additional secondary images that may be used for stereo matching to provide 3D images or other depth information. Each of the primary and secondary imagers 154, 104 includes dedicated pixels. Each imager 154, 104 may have its own respective image sensor or may share a common image sensor 102 with the other imagers as part of a contiguous assembly (as illustrated in the example of FIG. 11). The primary imager 154 can include a lens stack 156 disposed over the photosensitive regions of the sensor 102. The lens stack 156 can be placed in a lens barrel 158. In some cases, the primary imager 154 includes an IR-cut filter 160 disposed, for example, on a transmissive window such as a cover glass 162. The IR-cut filter 160 can be designed to filter substantially all IR light such that almost no IR light reaches the photosensitive region of the sensor 102 associated with the primary optical channel. Thus, in some cases, the IR-cut filter may allow only visible light to pass.

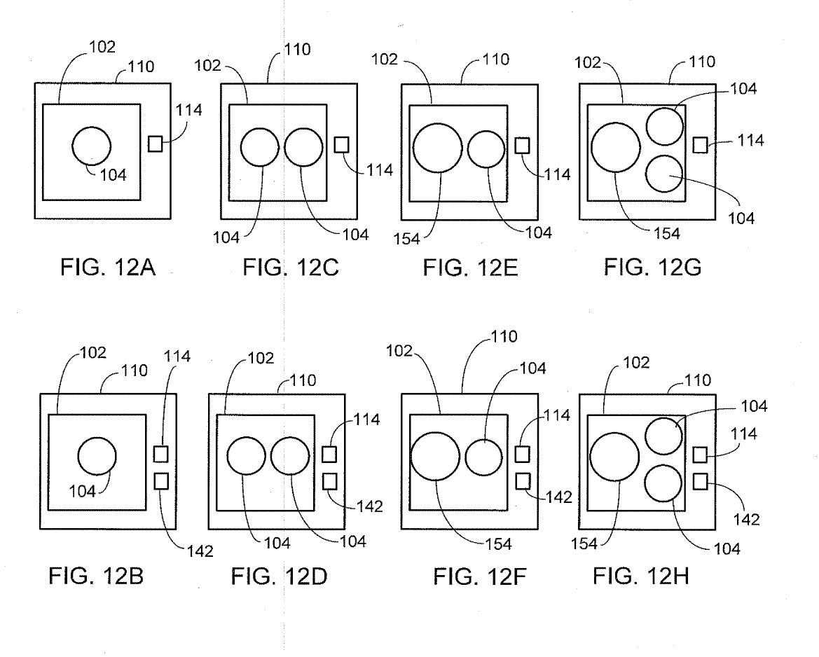

FIGS. 12A-12H illustrate schematically the arrangement of various optical modules. Each module includes at least one imager 104 that can be used for proximity sensing. Some modules include more than one imager 104 or 154 (see, e.g., FIGS. 12C, 12D, 12G, 12H). Such modules can be operable for both proximity sensing as well as imaging (including, in some cases, 3D stereo imaging). Further, some modules include a primary high-resolution imager 154 in addition to one or more secondary imagers 104 (see, e.g., FIGS. 12E-12H). Such modules also can provide proximity sensing as well as imaging. As described above, some modules may include a single light source 114 that generates coherent, directional, spectrally defined collimated light (see, e.g., FIGS. 12A, 12C, 12E, 12G). In other cases, the module may include multiple light sources 114, 142, one of which emits collimated light and another of which generates structured light (see, e.g., FIGS. 12B, 12D, 12F, 12H).

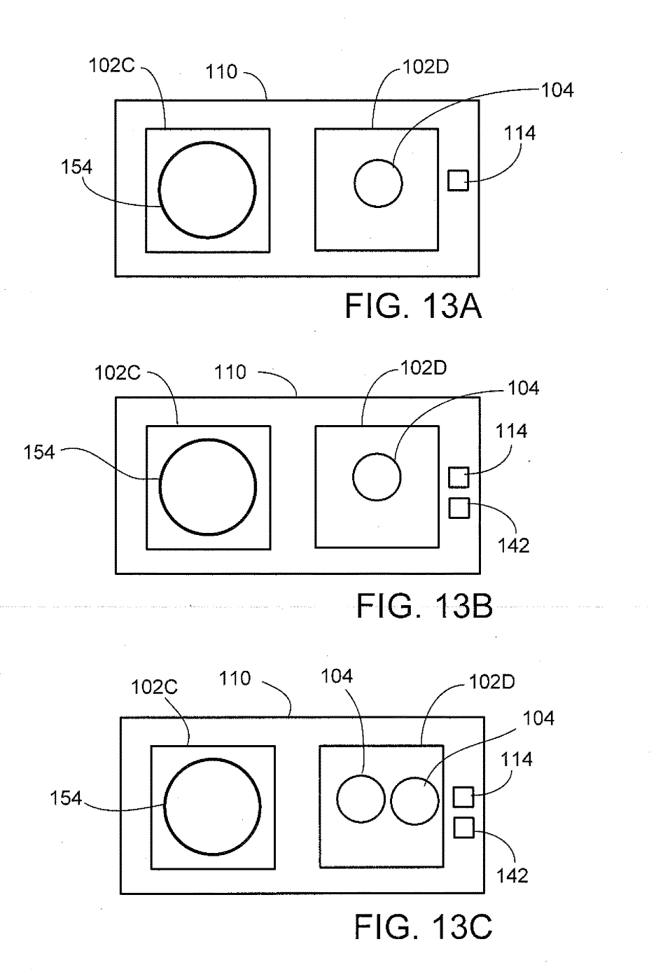

In the examples illustrated in FIGS. 12E-12H, the primary high-resolution imager 154 and the secondary imager(s) 104 are implemented using different regions of a common image sensor 102. However, in some implementations, the primary imager 154 and secondary imager(s) 104 may be implemented using separate image sensors 102C, 102D mounted on a common PCB 110 (see FIGS. 13A-13C). Each module may include one or more secondary imagers 104. Further, each module can include a single light source 114 that generates collimated light (see, e.g., FIG. 13A) or multiple light sources 114, 142, one of which emits a single beam of collimated light and another of which generates structured light (see, e.g., FIGS. 13B-13C). Other arrangements are possible as well.

The processing circuitry 112 can be configured to implement a triangulation technique to calculate the proximity of an object 124 in any of the foregoing module arrangements (e.g., FIGS. 12A-12H and 13A-13C). Further, for modules that include more than one imager (12C-12H and 13A-13C), the processing circuitry 112 can be configured to use a stereo matching technique for 3D imaging of an object 124.

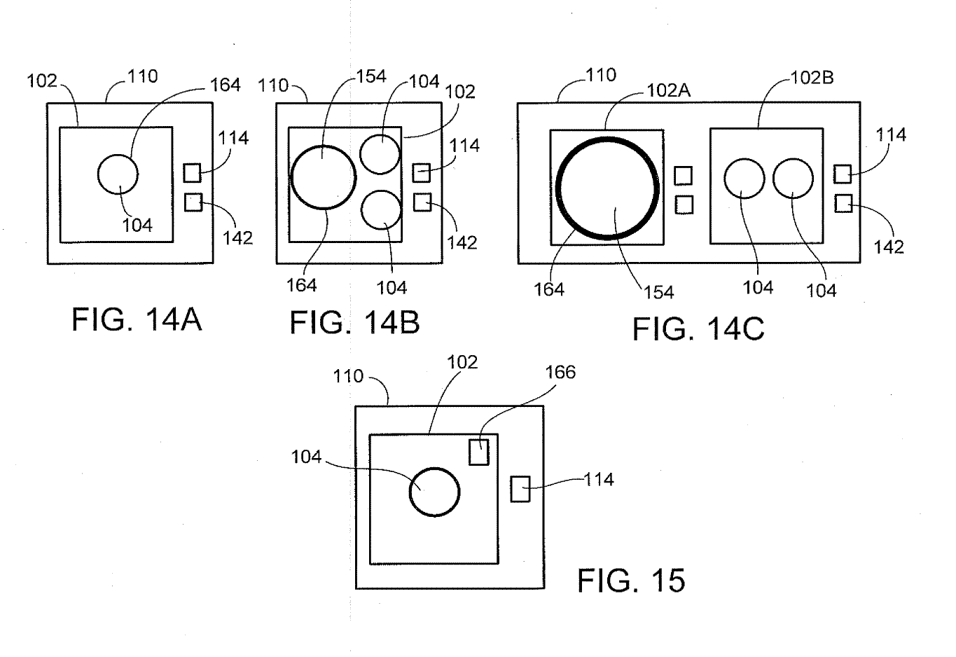

Some implementations include an autofocus assembly 164 for one or more of the optical channels. Examples are illustrated in FIGS. 14A-14C. In some instances, proximity data obtained in accordance with any of the techniques described above can be used in an autofocus assembly 164 associated with one of the module's optical channels. In some cases, proximity data can be used in an autofocus assembly associated with an imager or optical channel that is external to the module that obtains the proximity data.

Also, in some implementations, as shown in FIG. 15, some of the pixels of the image sensor 102 can be dedicated to an ambient light sensor (ALS) 166. Such an ALS can be integrated into any of the arrangements described above. In situations in which the primary and secondary imagers 154, 104 are provided on separate image sensors (e.g., FIG. 13A-13C or 14C), the ALS 166 can be provided, for example, on the same image sensor as the secondary imager(s).

As noted above, in some implementations, the different light sources 114, 142 may be operable at different powers from one another such that they emit different optical intensities from one another. This can be advantageous to help reduce the overall power consumption in some cases.

In some implementations, control circuitry 113 mounted on the PCB 110 (see FIGS. 1 and 11) can provide signals to the various components in the module to cause the module to operate selectively in a high-power or a low-power mode, depending on the type of data to be acquired by the module. For example, window-of-interest (windowing) operations can be used to read and process data only from selected pixels in the image sensor 102. Thus, power consumption can be reduced by reading and processing data only from selected pixels (or selected groups of pixels) instead of reading and processing all of the pixels. For example, in a multi-channel module, when only proximity sensing data is to be acquired, the window-of-interest would include the pixels within the area of the sensor under the secondary channel(s) 104. Data from all other pixels that are not selected would not need to be read and processed. Thus, the module can provide spatially dynamic power consumption, in which different regions of the sensor 102 are operated at different powers. In some cases, this can result in reduced power consumption. The control circuitry 113 can be implemented, for example, as a semiconductor chip with appropriate digital logic and/or other hardware components (e.g., digital-to-analog converter; microprocessor). The control circuitry 113 is, thus, configured to implement the various functions associated with such circuitry.

As an example, in a low-power mode of operation, proximity data from the secondary imagers 104 can be read and processed. The proximity can be based on light emitted by a low-power light projector 114 and reflected by an object (e.g., a person's ear or hand). If 3D image data is not to be acquired, then, data from the primary imager 154 would not need to be read and processed, and the high-power light projector 142 would be off. On the other hand, when 3D image data is to be acquired, the module can be operated in a high-power mode in which the high-power light projector 142 is turned on to provide a structured light pattern, and data from pixels in the primary imager 154, as well as data from pixels in the secondary imager(s) 104, can be read and processed.

In some implementations, the optical channels used for proximity sensing also can be used for gesture sensing. Light emitted by the low-power projector 114, for example, can be reflected by an object 124 such as a user's hand. As the user moves her hand, the processing circuitry 112 can read and process data from the secondary imagers 104 so as to detect such movement and respond accordingly. Signals indicative of hand gestures, such as left-right or up-down movement, can be processed by the processing circuitry 112 and used, for example, to wake up the host device (i.e., transition the device from a low-power sleep mode to a higher power mode). Referring to FIG. 12H, 13C or 14B, even if the high-power light projector 142 is off (while the low-power light projector 114 is on for gesture or proximity sensing), image data still can be read and processed from the primary imager 154, in some cases, based on the ambient light.

In the foregoing implementations, the modules are operable to distinguish between signals indicative of a reflection from an object interest and signals indicative of a spurious reflection in the context of proximity sensing. However, similar arrangements and techniques also can be used for other reflective light sensing applications as well. In particular, the following combination of features also can be used in modules designed for reflectance pulse oximetry applications (e.g., to detect blood oxygen levels) and/or heart rate monitoring (HRM) applications: at least one collimated light source (e.g., a VCSEL), an image sensor including an array of spatially distributed light sensitive components (e.g., an array of pixels), and processing circuitry operable to read signals from the spatially distributed light sensitive components and to assign a first peak signal associated with a first one of the light sensitive components to a spurious reflection and to assign a second peak associated with a second one of the light sensitive components to a reflection from an object of interest. The signals (i.e., peaks) assigned to the object of interest then can be used by the processing circuitry 112 according to known techniques to obtain, for example, information about a person's blood oxygen level or heart rate.

Pulse oximeters, for example, are medical devices commonly used in the healthcare industry to measure the oxygen saturation levels in the blood non-invasively. A pulse oximeter can indicate the percent oxygen saturation and the pulse rate of the user. Pulse oximeters can be used for many different reasons. For example, a pulse oximeter can be used to monitor an individual's pulse rate during physical exercise. An individual with a respiratory condition or a patient recovering from an illness or surgery can wear a pulse oximeter during exercise in accordance with a physician's recommendations for physical activity. Individuals also can use a pulse oximeter to monitor oxygen saturation levels to ensure adequate oxygenation, for example, during flights or during high-altitude exercising. Pulse oximeters, for example, can include processing circuitry to determine oxygen saturation and pulse rate and can include multiple light emitting devices, such as one in the visible red part of the spectrum (e.g., 660 nm) and one in the infrared part of the spectrum (e.g., 940 nm). The beams of light are directed toward a particular part of the user's body (e.g., a finger) and are reflected, in part, to one or more light detectors. The amount of light absorbed by blood and soft tissues depends on the concentration of hemoglobin, and the amount of light absorption at each frequency depends on the degree of oxygenation of the hemoglobin within the tissues.

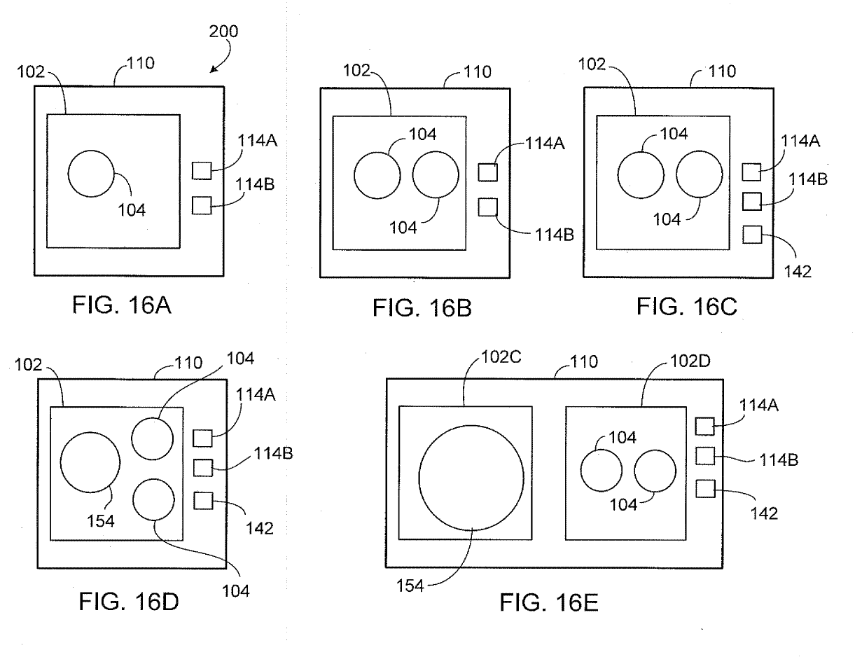

An example of an arrangement for a reflectance pulse oximetry module 200 is illustrated in FIG. 16A, which includes first and second light projectors 114A, 114B (e.g., VCSELs). The light projectors 114A, 114B are configured such that a greater amount of light from one projector is absorbed by oxygenated blood, whereas more light from the second projector is absorbed by deoxygenated blood. For example, the first light projector 114A can be arranged to emit light of a first wavelength (e.g., infra-red light, for example, at 940 nm), whereas the second light projector 114B can be arranged to emit light of a second, different wavelength (e.g., red light, for example, at 660 nm). When light emitted by the projectors 114A, 114B is directed toward a person's finger (or other part of the body), some of the light is absorbed and some of the light is reflected toward the image sensor 104, which includes spatially distributed light sensitive components (i.e., pixels) and which is sensitive to light at wavelengths emitted by each of the light projectors 114A, 114B.

Processing circuitry in the modules of FIGS. 16A-16E is operable to assign one or more first peak signals associated with a first one of the light sensitive components to a spurious reflection (e.g., a reflection from a transmissive window of the oximeter or other host device) and to assign one or more second peak signals associated with a second one of the light sensitive components to a reflection from the person's finger (or other body part) (see the discussion in connection with FIG. 2). The signals assigned to reflections from the object of interest (e.g., the person's finger) then can be used by the processing circuitry 112, according to known techniques, to determine the blood oxygen level. For example, the processing circuitry 112 can determine the blood oxygen level based on a differential signal between the off-line wavelength that exhibits low scattering or absorption and the on-line wavelength(s) that exhibits strong scattering or absorption.

In some cases, the pulse oximeter module includes more than one imager 104 (see FIG. 16B). The module also may include a light projector 142 that projects structured light (FIGS. 16C, 16D, 16E). In some instances, the module includes a primary imager 154, which may be located on the same image sensor 102 as the secondary imagers 104 (see, e.g., FIG. 16D) or on a different image sensor 102D (see, e.g., FIG. 16D). Such arrangements can allow the same module to be used for reflectance pulse oximetry applications as well as stereo imaging applications. In some implementations, the arrangement of FIGS. 16A-16E can be used both for reflectance pulse oximetry applications as well as proximity sensing applications. In such situations, at least one of the light projectors (e.g., 114A) and one of the imagers (e.g., 104) can be used for both the reflectance pulse oximetry as well as the proximity sensing applications.

Each of the module arrangements of FIGS. 16A-16E also can be used for heart rate monitoring (HRM) applications. In contrast to reflective pulse oximetry applications, however, only one light projector that emits light at a wavelength that can be absorbed by blood is needed (e.g., projector 114A). When used as a HRM module, some of the light emitted by the light projector 114A may encounter an arterial vessel where pulsatile blood flow can modulate the absorption of the incident light. Some of the unabsorbed light reflected or scattered from the arterial vessel may reach and be detected by the imager(s) 104. Based on the change in absorption with time, an estimate of the heart rate may be determined, for example, by the processing circuitry 112. When used in HRM applications, the processing circuitry 112 is operable to read signals from the imager(s) 104 and to assign a first peak signal associated with a first one of the light sensitive components to a spurious reflection (e.g., reflections from a transmissive window of a host device) and to assign a second peak associated with a second one of the light sensitive components to a reflection from a person's finger (or other body part) (see the discussion in connection with FIG. 2). The signals assigned to reflections from the object of interest (e.g., the person's finger) then can be used by the processing circuitry 112, according to known techniques, to estimate the person's heart rate.

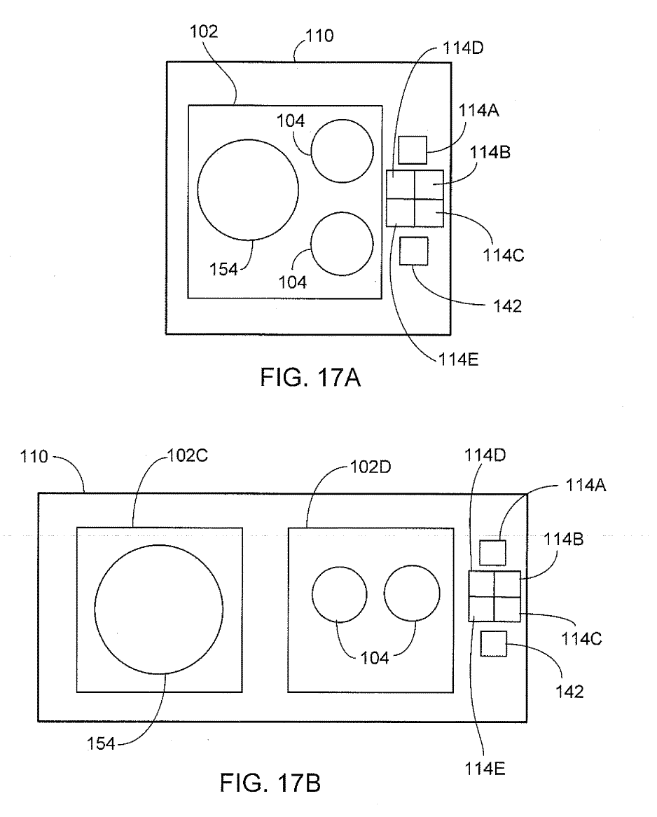

In some implementations, as shown in FIGS. 17A and 17B, additional light projectors operable to emit light of various wavelengths can be provided near the light projector 114B. The light projectors 114B, 114C, 114D and 114E may emit, for example, red, blue, green and yellow light, respectively. In some cases, the light projectors 114B-114E can be used collectively as a flash module, where the color of light generated by the flash module is tuned depending on skin tone and/or sensed ambient light. Thus, control circuitry 113 can tune the light from the projectors 114B-114E to produce a specified overall affect. Further, by placing the light projectors 114B 114E near the primary and secondary channels 154, 104 and the infra-red light projector 114A, the red light projector 114B also can be used for reflectance oximetry applications as described above. Additionally, in some cases, the individual light projectors 114B-114E can be activated individually by the control circuitry 113 to serve as visual indicators for the occurrence of various pre-defined events (e.g., to indicate receipt of an incoming e-mail message, to indicate receipt of a phone call, or to indicate low battery power). When operated in the indicator mode, the light projectors 114B-114E can use less power than when operated in the flash mode. The light projectors 114B-114E can be implemented, for example, as LEDs, laser diodes, VCSELs or other types of light emitters. Control circuitry 113 (see FIG. 1) can provide signals to turn on and off the various light projectors 112A-112E in accordance with the particular selected mode.

In view of the foregoing description, a single module can be used for one or more of the following applications: proximity sensing, gesture sensing, heart rate monitoring, reflectance pulse oximetry, flash and/or light indicators. For proximity sensing and heart rate monitoring applications, only a single light projector is needed, although in some cases, it may be desirable to provide multiple light projectors. For pulse oximetry applications, a second light projector can be provided as well. The processing circuitry 112 and control circuitry 113 are configured with appropriate hardware and/or software to control the turning on/off of the light projector(s) and to read and process signals from the imagers. In each case, the processing circuitry 112 can use the techniques described above to distinguish between spurious signals (e.g., signals indicative of reflections caused by a smudge on a cover glass) and signals of interest (e.g., signals indicative of reflections from an object whose proximity is to be determined, or a person's finger or other body part, in the case of heart rate monitoring and/or reflectance pulse oximetry applications). In some implementations, the module can be used for stereo imaging in addition to one or more of the foregoing applications. The addition of a light projector that provides structured light can be advantageous, for example, in some imaging applications.

Any of the foregoing module arrangements also can be used for other applications, such as determining an object's temperature. For example, if the imagers 104 are sensitive to infra-red light, the intensity of the detected signals can be indicative of the temperature (i.e., a higher intensity indicates a higher temperature). The processing circuitry 112 can be configured to determine the temperature of a person or object based on signals from the imagers using known techniques. Although light from the projector(s) is not required for such applications, in some cases, light from the projector (e.g., 114B) may be used to point to the object whose temperature is to be sensed.

Any of the foregoing module arrangements also can be used for determining an object's velocity. For example, the processing circuitry 112 can use signals from the imager(s) to determine an object's proximity as a function of time. In some cases, if it is determined by the processing circuitry 112 that the object is moving away from the module, the control circuitry 113 may adjust (e.g., increase) the intensity of light emitted by the structured light projector 142.

In some implementations, the foregoing modules may include user input terminal(s) for receiving a user selection indicative of the type of application for which the module is to be used. The processing circuitry 112 would then read and process the signals of interest in accordance with the user selection. Likewise, the control circuitry 113 would control the various components (e.g., light projectors 114) in accordance with the user selection.

In general, the module's light projector(s) in the various implementations described above should be optically separated from the imagers such that the light from the light projector(s) does not directly impinge on the imagers. For example, an opaque wall or other opaque structure can separate the light projector(s) from the imager(s). The opaque wall may be composed, for example, of a flowable polymer material (e.g., epoxy, acrylate, polyurethane, or silicone) containing a non-transparent filler (e.g., carbon black, a pigment, an inorganic filler, or a dye).

Other implementations are within the scope of the claims.

1. An optoelectronic module comprising:
a light projector operable to project light out of the module;
an image sensor including spatially distributed light sensitive components that are sensitive to a wavelength of light emitted by the light projector; and
processing circuitry operable to read signals from the spatially distributed light sensitive components of the image sensor and to assign a first peak signal associated with a first one of the light sensitive components to a spurious optical reflection and to assign a second peak signal associated with a second one of the light sensitive components to an optical reflection from an object of interest.

2. The module of claim 1 wherein the processing circuitry is further operable to use a position of the second light sensitive component to determine a distance to the object of interest.

3. The module of claim 2 wherein the processing circuitry is operable to use a triangulation technique to determine the distance to the object.

4. The module of claim 2 wherein the processing circuitry is operable to reference a look-up table or calibration data stored in memory to determine the distance to the object.

5.-7. (canceled)

8. The module of claim 1 including an optical channel over the image sensor, wherein the light projector is operable to emit collimated light at an angle relative to an optical axis of the optical channel.

9. The module of claim 1 including an optics assembly over the image sensor, wherein an optical axis of the module's optical detection channel is tilted at an angle with respect to a line that is perpendicular to a surface of the image sensor.

10. (canceled)

11. The module of claim 1 having a field of view for light detection, the field of view being tilted at an angle with respect to a line that is normal to a surface of the image sensor.

12. The module of claim 1 wherein the processing circuitry further is operable to process signals from the spatially distributed light sensitive components of the image sensor to obtain an image of the object.

13. The module of claim 1 wherein the spatially distributed light sensitive components of the image sensor are associated with a first optical channel, the module further including additional spatially distributed light sensitive components associated with a second optical channel.

14. The module of claim 13 wherein the first optical channel has a baseline distance that differs from a baseline distance of the second optical channel, wherein the baseline distances are measured with respect to the light projector.

15. The module of claim 13 wherein the processing circuitry is operable to use signals from the spatially distributed light sensitive components of the image sensor associated with the first optical channel to determine a proximity of an object within a first distance range and to use the additional spatially distributed light sensitive components associated with the second optical channel to determine a proximity of an object within a second distance range.

16. The module of claim 13, wherein the spatially distributed light sensitive components associated with the first optical channel and the spatially distributed light sensitive components associated with the second optical channel are operable to acquire data representing respective images of the object, and wherein the processing circuitry is operable to obtain depth data based on the acquired data.

17. The module of claim 16 wherein the processing circuitry is operable to obtain depth data for the images based at least in part on stereo matching.

18. The module of claim 16 wherein the processing circuitry is operable to obtain depth data for the images based at least in part on triangulation.

19. The module of claim 13 wherein the processing circuitry is operable to apply proximity data to an autofocus assembly associated with one of the optical channels, wherein the proximity data is based at least in part on signals from the image sensor.

20. The module of claim 13 wherein the processing circuitry is operable to apply proximity data to an autofocus assembly associated with an imager or optical channel that is external to the module, wherein the proximity data is based at least in part on signals from the image sensor.

21. The module of claim 1 further including a second light projector operable to generate structured light that is projected from the module.

22. The module of claim 21 operable such that at least some of the structured light generated by the second light projector is reflected by the object and sensed by the spatially distributed light sensitive components of the image sensor, and wherein the processing circuitry is operable to use a triangulation technique to determine the distance to the object based at least in part on signals generated by the spatially distributed light sensitive components of the image sensor in response to sensing the light reflected by the object.

23. The module of claim 21 operable such that at least some of the structured light generated by the second light projector is reflected by the object and sensed by the spatially distributed light sensitive components of the image sensor, and wherein the processing circuitry is operable to match pixels in stereo images based on texture provided by the structured light.

24. The module of claim 1 wherein the image sensor includes additional light sensitive components dedicated for ambient light sensing.

25. The module of claim 1 wherein the processing circuitry is operable to determine a heart rate based at least in part on the second peak signal.

26. The module of claim 1 further including a second light projector, wherein each light projector is operable to emit light of a different wavelength from the other light projector, and wherein the processing circuitry is operable to read signals from the light sensitive components and to assign some peak signals to spurious optical reflections and to assign other peak signals to optical reflections from an object of interest, the processing circuitry being further operable to determine a blood oxygen level based at least in part on the peak signals assigned to optical reflections from the object of interest.

27.-43. (canceled)
